(19) United States
(12) Patent Application Publication
(10) Pub. No.: US 2020/0129058 A1
(43) Pub. Date: Apr. 30, 2020

(54) VISUAL FIELD EXAMINATION DEVICE AND DETECTION METHOD

(71) Applicant: Chinese Academy of Medical Sciences & Peking Union Medical College, Institute of Biomedical Engineer

(72) Inventors: Yingxin LI, Tianjin (CN); Peng LI, Tianjin (CN); He HUANG, Tianjin (CN); Xiaogang CHEN, Tianjin (CN); Huijuan YIN, Tianjin (CN); Xuezhai ZHANG, Tianjin (CN)

(73) Assignee: Chinese Academy of Medical Sciences & Peking Union Medical College, Institute of Biomedical Engineer

(21) Appl. No.: 16/535,650

(22) Filed: Aug. 8, 2019

(30) Foreign Application Priority Data
Oct. 24, 2018 (CN) 201811240251.1
Feb. 21, 2019 (CN) 201910130049.1

(51) Int. Cl. A61B 3/024; A61B 3/00; G02B 27/01

(52) U.S. Cl. CPC A61B 3/024; A61B 3/0008; A61B 3/18; A61B 3/0033; G02B 27/017

(57) ABSTRACT

The present invention discloses a visual field examination device and detection method. The device includes: a virtual reality eye mask, electrodes, a visor, and an upper computer; the visor is located inside the virtual reality eye mask, for detecting one eye or both eyes; the electrodes are used for acquiring brain waves of a human body; the virtual reality eye mask is connected to the electrodes, for determining the scope of visual field according to the brain waves; the upper computer is connected to the virtual reality eye mask, for collecting a brain wave signal and a visual field scope signal. The method of the present invention greatly improves the efficiency of visual field examination, avoids the influence of a person's subjective response, and makes visual field screening become an intelligent detection instrument that can be used at home, without the needs of special operation and response from the detected person.

This application claims priority to Chinese application number 201811240251.1 filed on Oct. 24, 2018 and 201910130049.1 filed on Feb. 21, 2019. The above-mentioned patent application is incorporated herein by reference in its entirety.

### TECHNICAL FIELD

The present invention relates to the field of visual field detection, and in particular, to a visual field examination device and detection method.

### BACKGROUND

Now in ophthalmic visual field examination, various types of perimeters are generally used, including central perimeters, full perimeters, and the like; the detection method requires the detected person to respond during the detection, and the relevant test procedure is based on the response of the detected person to evaluate and judge the visual field of the detected person.

But this method has the following problems.

1) Long detection time. During visual field examination, affected by the scope of examination, the larger the scope of examination is, the more stimulating points are examined, and as the detected person is required to respond actively, the time needed for the measurement is longer with the test times of the stimulating points. The long-time test makes the detected person fatigued, and the test results are affected.

2) Great subjective influence of the detected person. Traditional visual field examination requires the detected person to respond after seeing a stimulating light flash, so as to judge the visual field of the detected person by the response. In a first aspect, the detected person may deliberately not respond after seeing a stimulating point or deliberately respond without seeing the stimulating point. In a second aspect, if the detected person is distracted, the results are often affected. In a third aspect, even if the detected person cooperates well, the detection results may be affected by the factor of differences, like the age of the detected person.

3) Inconvenience. First, the existing perimeters are relatively large in size and heavy in weight, and require a special lifting platform when necessary. Second, the detection of the perimeters requires the operation of a doctor and the cooperation of the detected person, and still requires the doctor to observe the attention of the detected person during detection, so as to guarantee the accuracy of the detection results. Third, professional trainings are necessary for use of the perimeters; general users are not capable of using such equipment to achieve visual field detection. Fourth, as the detection time is long, visual field detection needs to wait for a long time.

### SUMMARY

An objective of the present invention is to provide a visual field examination device and detection method, which greatly improve the efficiency of visual field examination, improve the accuracy thereof, and avoid the influence of subjective response.

To achieve the foregoing objective, the present invention provides the following solution.

A visual field examination device, where the device includes: a virtual reality eye mask, electrodes, a visor, and an upper computer; the visor is located inside the virtual reality eye mask, for detecting one eye or both eyes; the electrodes are used for acquiring brain waves of a human body; the virtual reality eye mask is connected to the electrodes, for determining the scope of visual field according to the brain waves; the upper computer is connected to the virtual reality eye mask, for collecting a brain wave signal and a visual field scope signal.

Optionally, the virtual reality eye mask includes an eye mask body and an eyeglass frame body; the eyeglass frame body is located inside the eye mask body; the visor is located inside the eyeglass frame body.

Optionally, the electrodes include positive electrodes and negative electrodes; one of the positive electrodes and one of the negative electrodes constitute an electrode group, and there are four electrode groups.

Optionally, the upper computer is an industrial personal computer.

Optionally, the upper computer and the virtual reality eye mask are in wired connection.

Optionally, the upper computer and the virtual reality eye mask are in wireless connection.

Optionally, the device further includes: a voice prompting module, connected to the upper computer.

To achieve the foregoing objective, the present invention provides the following solution.

A visual field detection method, where the method includes:

lighting a stimulating point in a pulse mode;

obtaining an electro-ocular signal waveform acquired by electrodes, according to the stimulating point;

determining whether the electro-ocular signal waveform is the same as a standard electro-ocular signal waveform;

if yes, determining that the visual field is complete; and if not, determining that the visual field is defective.

According to the specific embodiments of the present invention, the present invention discloses the following technical effects: the present invention provides a visual field examination device, where the device includes: a virtual reality eye mask, electrodes, a visor, and an upper computer; the visor is located inside the virtual reality eye mask, for detecting one eye or both eyes; the electrodes are used for acquiring brain waves of a human body; the virtual reality eye mask is connected to the electrodes, for determining the scope of visual field according to the brain waves; the upper computer is connected to the virtual reality eye mask, for collecting a brain wave signal and a visual field scope signal. The method of the present invention greatly improves the efficiency of visual field examination, improves the accuracy thereof, avoids the influence of a person's subjective response, and makes visual field screening become an intelligent detection instrument that can be used at home, without the needs of special operation and response from the detected person.

### BRIEF DESCRIPTION OF THE DRAWINGS

To describe the technical solutions in the embodiments of the present invention or in the prior art more clearly, the following briefly introduces the accompanying drawings required for describing the embodiments. Apparently, the accompanying drawings in the following description show merely some embodiments of the present invention, and a person of ordinary skill in the art may still derive other drawings from these accompanying drawings without creative efforts.

FIG. 1 is a schematic diagram of a visual field examination device according to an embodiment of the present invention; and

FIG. 2 is a flow chart of a visual field detection method according to an embodiment of the present invention.

### DETAILED DESCRIPTION

The following clearly and completely describes the technical solutions in the embodiments of the present invention with reference to the accompanying drawings in the embodiments of the present invention. Apparently, the described embodiments are merely a part rather than all of the embodiments of the present invention. All other embodiments obtained by a person of ordinary skill in the art based on the embodiments of the present invention without creative efforts shall fall within the protection scope of the present invention.

To make the foregoing objective, features, and advantages of the present invention clearer and more comprehensible, the present invention is further described in detail below with reference to the accompanying drawings and specific embodiments.

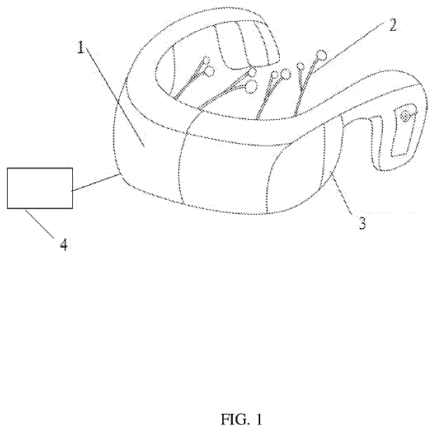

FIG. 1 is a schematic diagram of a visual field examination device according to an embodiment of the present invention. As shown in FIG. 1, a visual field examination device, where the device includes: a virtual reality eye mask 1, electrodes 2, a visor 3, and an upper computer 4; the visor 3 is located inside the virtual reality eye mask 1 to form a complete darkroom for a single eye when necessary, and is used for detecting one eye or both eyes simultaneously; the electrodes 2 are directly attached to a head collection part, and are used for acquiring brain waves of a human body; the virtual reality eye mask 1 is connected to the electrodes 2, and is used for determining the scope of visual field according to the brain waves; the upper computer 4 is connected to the virtual reality eye mask 1, and is used for collecting a brain wave signal and a visual field scope signal.

The virtual reality eye mask 1 includes an eye mask body and an eyeglass frame body; the eyeglass frame body is located inside the eye mask body; the visor is located inside the eyeglass frame body.

The electrodes 2 include positive electrodes and negative electrodes; one of the positive electrodes and one of the negative electrodes constitute an electrode group, and there are four electrode groups.

The upper computer 4 is an industrial personal computer. The upper computer 4 and the virtual reality eye mask are in wired or wireless connection.

The visual field examination device further includes a voice prompting module, connected to the upper computer, for providing an auxiliary voice prompting function.

The virtual reality eye mask has wings on the left and right sides, and has a soft internal structure, which can meet the requirement of beam blocking; the hole positions of the eye mask are slightly larger than adult glasses to improve the shading performance.

The present invention adopts a closed virtual reality (VR) eye mask, and the image of detection software can be directly displayed on the inner side of the eye mask; the detection software can be switched to simulate the really tested visual field distances and bright point stimulation of different examination strategies; the software can be a platform system for diagnosis and treatment of different ophthalmic diseases.

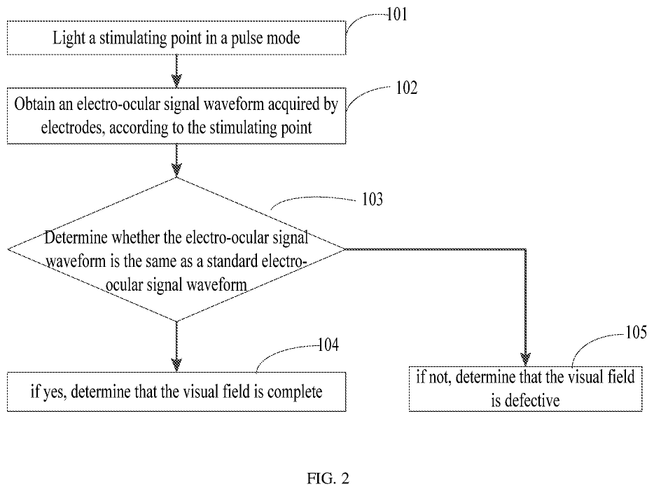

FIG. 2 is a flow chart of a visual field detection method according to an embodiment of the present invention. As shown in FIG. 2, a visual field detection method, where the method includes:

Step 101: light a stimulating point in a pulse mode.

Step 102: obtain an electro-ocular signal waveform acquired by electrodes, according to the stimulating point, specifically:

each stimulating point is lighted in a pulse mode; normally, the visual cortex responds to generate an eye point signal, which is automatically recorded by a software system; then the next stimulating point is lighted, or the same stimulating point is lighted with different brightness, and is continuously automatically recorded by the software system; after the stimulating points are lighted, any electro-ocular signal waveforms generated by normal vision, generated by abnormal vision, or generated without vision are all correspondingly automatically recorded by the software system.

Step 103: determine whether the electro-ocular signal waveform is the same as a standard electro-ocular signal waveform; the software automatically evaluates whether the electro-ocular feedback signal waveform corresponding to the brightness of the stimulating points is different from the threshold value of the standard electro-ocular signal waveform, to determine whether the visual field is defective.

Step 104: if yes, determine that the visual field is complete.

Step 105: if not, determine that the visual field is defective.

The virtual reality eye mask (VR eye mask) is used to achieve a variety of visual field detection modes, including central visual field detection, peripheral visual field detection, visual field destructive screening, and the like; the VR technology is applied to simulate real visual field distance and bright point stimulation; the electro-ocular signals are picked up by the response of the visual cortex to the bright points. The method of the present invention replaces a traditional manual button method, reducing and avoiding response errors from manual work; by matching the flashing time of the stimulating points with the acquired electro-ocular signals, fast visual field examination is achieved, and short-time visual field evaluation becomes possible.

As any electro-ocular signal waveforms generated by normal vision, generated by abnormal vision, or generated without vision are all correspondingly automatically recorded by the software system, the present invention can automatically generate a topographic map of visual field to provide a basis for diagnosis. In addition, since the data of optic nerves in corresponding time is provided, the present invention can provide objective data for the diagnosis of other diseases.

Through the application of the present invention, the efficiency of visual field examination can be greatly improved, the accuracy thereof can be improved, and the influence of subjective response can be avoided. Visual field screening becomes an intelligent detection instrument that can be used at home, without the needs of special operation and response from the detected person; and daily visual field detection (glaucoma prevention and visual field screening) and tracking management of glaucoma patients also become possible, which can improve the management level of glaucoma patients and prevent the glaucoma patients from becoming blind.

The present invention can realize rapid evaluation of the whole visual field through different examination strategies. The present invention can serve as a platform system, which can be designed as treatment software for exercise of visual diseases, for the treatment of such ophthalmic diseases as myopia, amblyopia and ametropia. The present invention can also be designed as test software for diseases like myopia, for the diagnosis of diseases like myopia, to achieve integration of various device functions. The present invention no longer needs fixation, but performs detection stimulation according to the position of the eyeball in real time, reducing the requirement for the patient's cooperation degree, and also reducing the time required for the visual field test and the burden on the patient and the doctor. The present invention can realize centralized monitoring and management of various ophthalmic diseases, thus reducing medical costs.

Each embodiment of the present specification is described in a progressive manner, each embodiment focuses on the difference from other embodiments, and the same and similar parts between the embodiments may refer to each other.

In this paper, several examples are used for illustration of the principles and embodiments of the present invention. The description of the foregoing embodiments is used to help illustrate the method of the present invention and the core principles thereof. In addition, those skilled in the art can make various modifications in terms of specific embodiments and scope of application in accordance with the teachings of the present invention. In conclusion, the content of the present specification shall not be construed as a limitation to the present invention.

What is claimed is:

1. A visual field examination device, wherein the device comprises: a virtual reality eye mask, electrodes, a visor, and an upper computer; the visor is located inside the virtual reality eye mask, for detecting one eye or both eyes; the electrodes are used for acquiring brain waves of a human body; the virtual reality eye mask is connected to the electrodes, for determining the scope of visual field according to the brain waves; the upper computer is connected to the virtual reality eye mask, for collecting a brain wave signal and a visual field scope signal.

2. The visual field examination device according to claim 1, wherein the virtual reality eye mask comprises an eye mask body and an eyeglass frame body; the eyeglass frame body is located inside the eye mask body; the visor is located inside the eyeglass frame body.

3. The visual field examination device according to claim 1, wherein the electrodes comprise positive electrodes and negative electrodes; one of the positive electrodes and one of the negative electrodes constitute an electrode group, and there are four electrode groups.

4. The visual field examination device according to claim 1, wherein the upper computer is an industrial personal computer.

5. The visual field examination device according to claim 1, wherein the upper computer and the virtual reality eye mask are in wired connection.

6. The visual field examination device according to claim 1, wherein the upper computer and the virtual reality eye mask are in wireless connection.

7. The visual field examination device according to claim 1, wherein the device further comprises: a voice prompting module, connected to the upper computer.

8. A visual field detection method, wherein the method comprises:
lighting a stimulating point in a pulse mode;
obtaining an electro-ocular signal waveform acquired by electrodes, according to the stimulating point;
determining whether the electro-ocular signal waveform is the same as a standard electro-ocular signal waveform;
if yes, determining that the visual field is complete; and
if not, determining that the visual field is defective.
